(19) United States
(12) Patent Application Publication
(10) Pub. No.: US 2014/0288415 A1
(43) Pub. Date: Sep. 25, 2014

(54) Imaging Method and Device for the Cardiovascular System

(71) Applicants: Leonardo Forzoni, Pistoia (IT); Magda Costi, Borgo San Lorenzo (FI) (IT)

(72) Inventors: Leonardo Forzoni, Pistoia (IT); Magda Costi, Borgo San Lorenzo (FI) (IT)

(73) Assignee: Esaote S.p.A., Genova (IT)

(21) Appl. No.: 14/217,739

(22) Filed: Mar. 18, 2014

(30) Foreign Application Priority Data
Mar. 19, 2013 (IT) GE2013A000032

(51) Int. Cl. A61B 5/02; A61B 5/06; A61B 8/08

(52) U.S. Cl. CPC A61B 5/02; A61B 8/0833; A61B 5/061
USPC 600/424; 600/407; 600/447

(57) ABSTRACT

An imaging method and device for the cardiovascular system, the method including the steps of:
acquiring a volumetric image having scan planes adjacent to each other of a periodically moving object;
identifying at least one time interval of at least one periodic movement of that object;
rearranging the scan planes of the volumetric image based on the time interval by combining with each other the scan planes that are in the same time position with respect to the time interval, the object including at least one blood vessel with periodic movements, at least constriction and dilation, and
generating and displaying a virtual endo-navigation three-dimensional model by segmenting and reconstructing the volumetric image, the point of view being movable along at least one path, for determining plaques, stenoses or strictures, and/or for analyzing moving structures inside the vessel such as valve leaflets.

### FIELD OF THE INVENTION

The present invention relates to an imaging method and device for the cardiovascular system, wherein the method includes the following steps:
acquiring a volumetric image comprising scan planes adjacent to each other of a periodically moving object;
identifying a time interval of a periodic movement of said object; and
rearranging the scan planes of the volumetric image on the basis of the time interval by combining with one another the scan planes that are in the same time position with respect to the time interval.

### BACKGROUND OF THE INVENTION

Imaging methods are known and used, which allow images of periodically moving objects to be acquired by performing evaluations that take the movement into consideration and therefore bring the volumetric images back to a condition without movement-related distortions.

An example of such methods is described in document U.S. Pat. No. 6,966,878, wherein an acquisition of a volumetric image of a moving object, which repeats a movement cycle over time, is periodically performed.

Therefore, a time interval of the periodic movement is identified within the volumetric scan, and the volumetric scan is rearranged based on the time interval.

That document describes the method known as Spatio-Temporal Imaging Correlation (STIC), wherein different periodic patterns over time are detected and temporally-spatially correlated events are reconstructed. Therefore, the identification of the time interval occurs by correlating the different scan planes.

In similar methods an external signal acquired contemporaneously with the image is used, as for example an electrocardiogram (ECG), which accurately indicates the time frames of the events involved in the periodic movement of the object for rearranging the scan planes in a process which is known as “triggering.”

The known methods are mainly intended for the imaging of the heart (three-dimensional fetal echocardiography the first one, three-dimensional adult echocardiography the second one), and provide a good investigation of the anatomy and of the physiology of the organ regardless of the movement to which it is periodically subjected.

On the contrary, when attention is paid to blood vessels (both arterial and venous vessels), the known methods are not sufficient for a good evaluation of possible anomalies.

For example, in regard to arteries, particularly the carotid artery, there is the unsatisfied need for a satisfactory three-dimensional morphologic evaluation made by the user about possible plaques or stenoses.

On the contrary, in regard to veins, for example the internal jugular vein, the known methods do not produce satisfactory results for the three-dimensional study of the lumen, of a possible presence of stenoses/strictures/compressions, or of a movement of the valve leaflets.

Veins particularly have periodic movements that depend on a plurality of reasons, among which heartbeat and breathing.

This is more evident above all in the study of the lumen of the internal jugular vein both at rest and when it changes based on different pressure maneuvers, such as the Valsalva maneuver, Müller maneuvers or deep breathing.

### SUMMARY OF THE INVENTION

The present invention aims at overcoming the limits of the prior art by a method, related to an object composed of at least one blood vessel with periodic movements, at least constriction and dilation, which includes the step of:
generating and displaying a virtual endo-navigation three-dimensional model by segmenting and reconstructing a volumetric image, the point of view being movable along at least one path, for determining plaques, stenoses, strictures and/or for analyzing moving structures inside the vessel.

The structures moving in the vessel can preferably be valve leaflets but also elements on the walls of the vessel, bifurcation portions, damaged portions, etcetera.

Providing a three-dimensional endo-navigation model with at least one path, on which the point of view is movable, is very advantageous for the substantially tubular shape of blood vessels.

In order to obtain the desired result, a perspective effect is generated by combining the image of the section plane, wherein the point of view lies with the images of the following section planes.

Therefore, the user has an investigation system available to her, by which possible anomalies, particularly constrictions, plaques or stenoses, or the presence of valve leaflets or their evolution in the position over time, are immediately recognizable after reconstructing the image.

In one embodiment, said identification of at least one time interval of at least one periodic movement of said blood vessel occurs by comparing said scan planes with each other and/or by contemporaneously acquiring an electrocardiogram signal and/or by contemporaneously acquiring a breathing signal.

The comparison of the scan planes with each other can be performed by methods conventionally used in STIC technology.

In particular, it is possible to provide a Fourier time analysis, or an autocorrelation of the signal intensity to be used.

The contemporaneous acquisition of an ECG signal and/or a breathing signal, on the contrary, can be used according to the standard methods of the “triggered” or “gated” type acquisitions.

The breathing signal can be acquired by any method known in the prior art, preferably by measuring the dilation of the trunk in the chest and/or abdomen area when breathing.

The described three methods can be used individually and as an alternative or in combination with each other, or in pairs or all at the same time.

The choice of the method or methods used is made based on what is desired to be displayed and of the anatomical district of interest.

The volumetric image obtained is subjected to a plurality of influences such as, for example, breathing, possible movements, and heartbeat for the volumetric reconstruction of the internal jugular vein. As regards the carotid artery, the image reconstruction can be influenced by the heartbeat and possible movements of the neck, due for example to unintentional movements, deglutition or other movements.

The STIC method, for example, can be particularly advantageous in the study of the movement of the valve leaflets of the internal jugular vein.

The acquisition of the breathing signal on the contrary is particularly advantageous in the study of the lumen of the internal jugular vein, both at rest and subjected to pressure maneuvers.

It is possible to combine the different methods for rearranging the scan planes through single parallel processing chains, which process the volumetric image independently from each other, the results being finally combined with each other in a single volumetric image by settable weighings.

As the reconstruction of the volumetric image is synchronized based on one or more of the following “triggering,” ECG, breath, or STIC, the user, once the final volume is obtained, can select which triggering has to be used as the main reconstruction trace by suitably weighing the contribution of each method or not considering the other ones.

In one embodiment, the contributions of comparing said scan planes one with the other and acquiring an electrocardiogram signal and acquiring a breathing signal in rearranging the scan planes are regulated by a weighed sum.

In another embodiment, said virtual endo-navigation three-dimensional model provides on the reconstructed image one or more virtual lighting points, the shadows being generated correspondingly to the positioning of said one or more lighting points and said one or more lighting points being movable and settable by the user.

The possibility of moving the virtual lighting points is particularly advantageous for studying the presence of plaques or stenoses in the arteries or for studying possible stenoses or the movement of the valve leaflets in the veins.

The modification of the shadows, can efficaciously point out possible constrictions or anomalies in the walls of the vessel.

The lighting point can be set a priori after generating the three-dimensional model but before moving the point of view, or it can be moved during the movement of the point of view.

In a preferred embodiment, the volumetric image is of the ultrasound type, although other types of imaging can be used.

In another embodiment, said acquisition is made by B-mode and/or Doppler mode.

The Doppler mode allows flows and speeds to be evaluated, which in turn follow periodic patterns.

In the presence of constrictions and anomalies, moreover the speed is subjected to changes that can be easily detected.

According to a further embodiment the point of view of said virtual endo-navigation is automatically movable, said path being determined by the user.

Therefore, once the three-dimensional model is generated and once the image is reconstructed, the user can trace one or more paths in the image, preferably in the blood vessel.

Therefore the point of view automatically continues along the predetermined path.

In another embodiment, starting from said three-dimensional model one or more section images along one or more predetermined section planes are generated and displayed, and the lumen of the blood vessel all along the extension of the blood vessel comprised in the volumetric image is calculated.

Preferably the section images are displayed besides the endo-navigation image, and the plane upon which the point of view lies is displayed if it is tangent to the section plane of the image.

As an alternative, it is possible to set one or more section planes in relation to the point of view, and the section images are generated during the movement of the point of view.

In one embodiment, the point of view of said virtual endo-navigation is automatically movable, said path being identified by the center of the lumen of the blood vessel all along the extension of the blood vessel comprised in the volumetric image.

Therefore, the lumen of the vessel all along its extension is calculated, and the center of the vessel lumen is calculated. Therefore the path of the point of view is defined by the set of the centers of the vessel lumen in the adjacent scan planes.

Once the path is automatically defined in this manner, the point of view is caused to automatically continue along such path.

Advantageously, it is possible to display a graph showing the lumen of the vessel in relation to the axial position in the three-dimensional model, for better evaluating the constrictions.

The acquisition of said volumetric image can be performed by a motorized 2D probe, wherein a transducer array is rotated in a fan-like acquisition.

A problem with this configuration is that the spatial resolution varies along the depth of the acquired volume.

In an advantageous embodiment, the acquisition of said volumetric image is performed by a parallel scanning probe.

This allows the acquisition of the volumetric image to be performed by the insonification and the reception of echoes in a sequential manner along adjacent scan planes.

In another embodiment, the probe has a flat emitting surface.

The probe performs a parallel scanning of a linear transducer such that it is able to reconstruct a volume represented by a parallelepiped having the width of the array of the probe and the length equal to the linear length of the scanning track. The depth (height) of the parallelepiped is given by the depth settings that are set such to have the segment of interest of the vessel inside the acquired volume.

The present invention also relates to an imaging device for the cardiovascular system comprising:
means acquiring a volumetric image comprising scan planes adjacent to each other of a periodically moving object;
means identifying at least one time interval of at least one periodic movement of said object;
means for rearranging said scan planes of said volumetric image based on said time interval, which rearranging means combine with each other the scan planes that are in the same time position with respect to said time interval, wherein said object is composed of at least one blood vessel with periodic movements, at least constriction and dilation, and the device further comprises:
means generating and displaying a virtual endo-navigation three-dimensional model by segmenting and reconstructing said volumetric image, wherein the point of view is movable along at least one path, for determining plaques, stenoses or strictures.

Preferably, the means acquiring a volumetric image comprise an ultrasound probe and a reception signal processing unit for generating images along adjacent scan planes.

In one preferred embodiment, said device is intended to operate according to the above described method.

### BRIEF DESCRIPTION OF THE DRAWINGS

These and other characteristics and advantages of the present invention will be clearer from the following description of a few embodiments shown in the enclosed drawings, wherein:

FIG. 1 is a general diagram of a method according to the present invention;

FIG. 2 is one embodiment of a device according to the present invention.

### DETAILED DESCRIPTION OF EMBODIMENTS OF THE INVENTION

Detailed descriptions of embodiments of the invention are provided herein. It is to be understood, however, that the present invention may be embodied in various forms. Therefore, the specific details disclosed herein are not to be interpreted as limiting, but rather as a representative basis for teaching one skilled in the art how to employ the present invention in virtually any detailed system, structure, or manner.

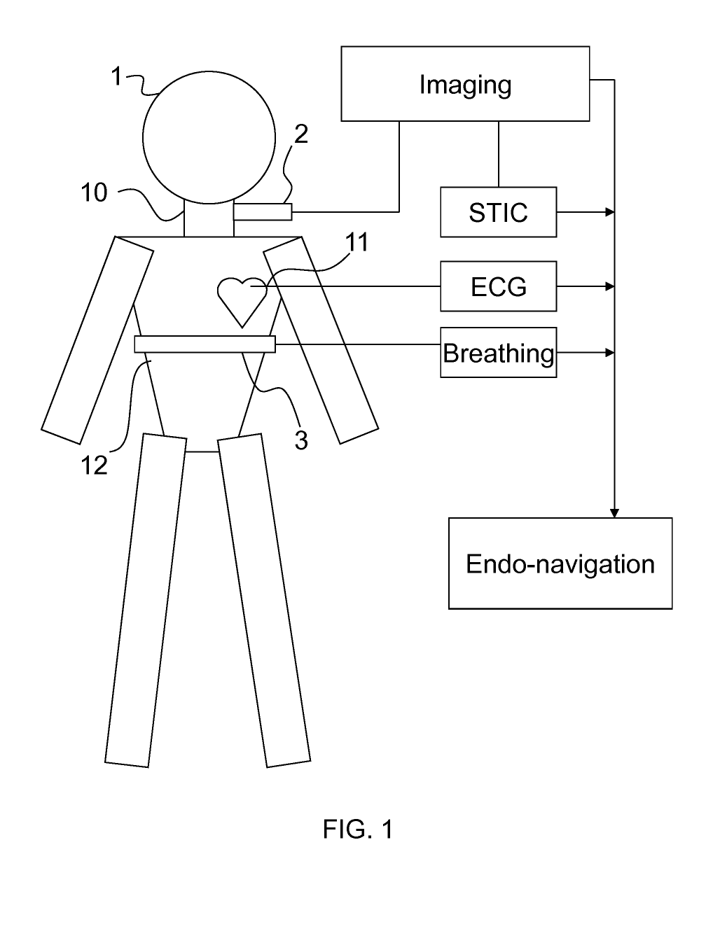

FIG. 1 shows a preferred and not limitative embodiment of the imaging method for the cardiovascular system, wherein the volumetric image is acquired from a patient 1, in the anatomical district of the neck 10, for example for imaging the common carotid artery or the internal jugular vein.

The method provides for acquiring a volumetric ultrasound image of an object by means of an ultrasound probe 2. The volumetric image comprises scan planes adjacent to each other.

The image can be acquired in the B-mode and/or in Doppler mode.

Therefore, at least one time interval of at least one periodic movement of the object is identified, particularly dilation and constriction movements, and the scan planes are rearranged based on the time interval by combining with each other the scan planes that are in the same time position with respect to the time interval.

The identification of the time interval occurs by using, as a combination or as an alternative, a STIC method wherein said scan planes are compared with each other, an ECG, or a breathing signal.

The breathing signal is acquired by means 3 measuring the dilation of the trunk 12 in the chest and/or abdomen area during the breathing, which measurement means 3 can be of any type currently known to a person skilled in the art.

Preferably the measurement means 3 comprise one or more straps that can be of the elastic or piezoelectric type, and can be arranged at the chest and/or abdomen area.

In the case of a single strap, it is preferred to place it at the chest area.

The described three methods can be used individually and as an alternative or in combination with each other, or in pairs or all together, and the choice of the method or methods used is made based on what is desired to be seen and of the anatomical district of interest.

For example, it is possible to consider only the ECG signal in the case of imaging the common carotid artery for a healthy patient.

On the contrary, it is advantageous to use the ECG method and the STIC method in a patient suffering from neurologic pathology, such to contemporaneously detect the heartbeat and possible unintentional movements, which in turn, the more they have a periodicity characteristic, the more they can be easily corrected.

The STIC method can easily detect voluntary and/or involuntary repetitive movements, for example, swallowing or tremor.

In the case of the internal jugular vein, on the contrary, the influence of the heartbeat and of breathing on the blood flow varies from patient to patient, such as in the same patient from the right side to the left side and also from a body position to a body position, for example supine or sitting position, in the same vein of the same patient.

In this case the characteristics of the flow are detected by the B-mode or by the Color Doppler or Power Doppler mode or by similar technologies for displaying the blood flow. The combination of “triggering” methods, suitably weighed, is then selected, which is the most suitable for reconstructing the volumetric image.

Once the image is reconstructed a virtual endo-navigation three-dimensional model is generated by suitably segmenting and reconstructing the volumetric image.

Therefore, a view of the three-dimensional model from a specific point of view is displayed, and such point of view is movable within the three-dimensional model along a path for determining plaques, stenoses or constrictions.

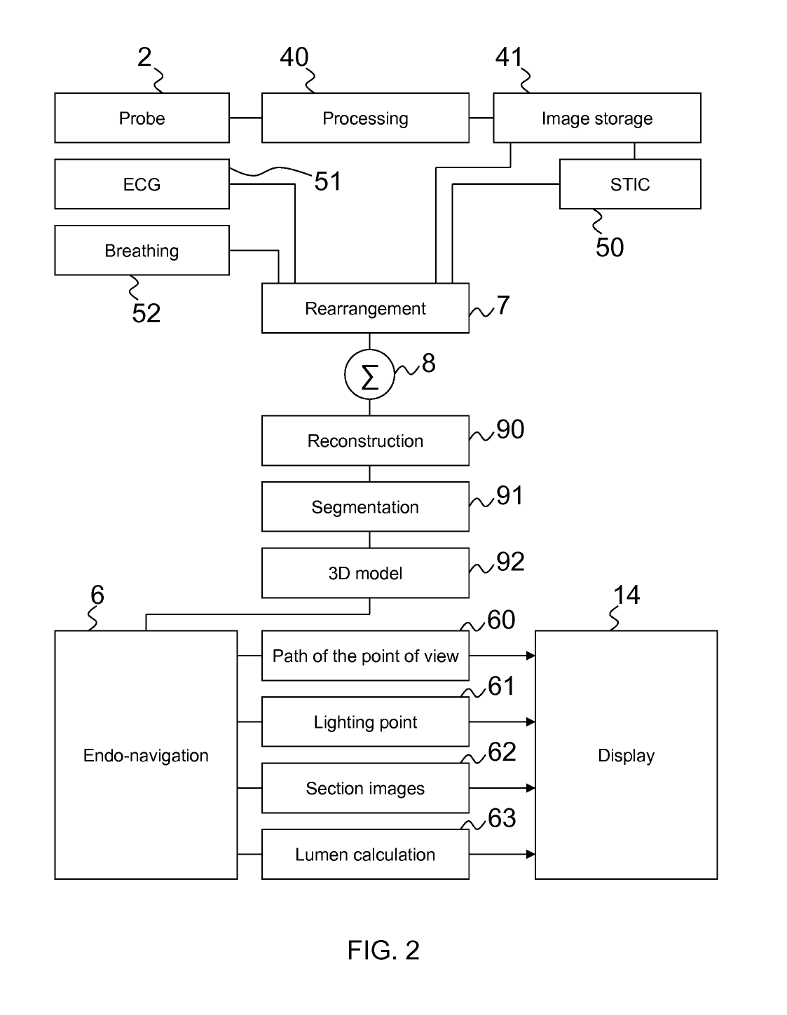

FIG. 2 shows the diagram of one embodiment of a device according to the present invention, wherein the boxes may be considered as a functional elements of the method.

The device includes an ultrasound probe 2 comprising a linear array of electroacoustic transducers for emitting ultrasound pulses in the body under examination and for receiving echoes generated by the body under examination; a system transmitting to the array electric signals generated by an emission signal generating unit, and a system transmitting from the array electric signals to a reception signal processing unit 40 for generating images along adjacent scan planes, and for storing such images in an image storage unit 41.

The probe 2 preferably is provided with a system for translating the array along an axis perpendicular to the longitudinal axis of the array from an initial position to a final position and preferably has a flat emitting surface.

In a preferred embodiment, the probe is provided with a pair of rails or tracks and the transducer array is arranged to translate along the rails during acquisition, in a direction perpendicular to its axis.

This parallel linear scanning probe is configured to create a parallelepiped volume, and the linear array of transducers acquires perfectly parallel planes characterized by high spatial resolution, which is non-geometrically dependent from the different spatial resolution due to depth and scanning angle.

Therefore, emissions and receptions are made for a plurality of scan planes during the translation, there being provided a system for combining the generated images in a single volumetric image.

Advantageously, the movement direction of the array coincides with the longitudinal axis of the blood vessel under examination.

As an alternative, it is possible to provide a two-dimensional probe wherein rows of different transducers adjacent to each other are sequentially and individually activated.

With this 3D linear matrix array, the acquisition is performed without the movement of any mechanical parts. The array elements are disposed in a matrix and scan the volume under examination by means of electronic steering. Also in this case, the acquired volume is a parallelepiped volume, with a small possible curvature due to the non-completely linear shape of the array.

As another alternative, a manual acquisition can be performed by a 2D linear probe in combination with a tracking system comprising a transmitter close to the patient and a receiver fixed to the probe.

The transmitter and receiver are preferably electromagnetic as described for example in document EP2706372.

The tracking system enables the combination of different 3D ultrasound volumes and the navigation within, on the basis of the positioning information detected by means of the electromagnetic field.

Two approaches can be used. In a first approach a global 3D reconstruction is performed, based only on the geometric and position information given by the probe position and orientation within the electromagnetic field. In a second approach, in addition to the information coming from the tracking system, a data analysis is performed focusing on tissue structure recognition, in order to find the best matching between the volumes. This second approach can be particularly useful to compensate little tissue compression by the ultrasound probe during scanning.

This method allows obtaining not only parallelepiped volumes but also other shapes.

There are provided a unit 51 for detecting an ECG signal, a unit 52 for detecting a breathing signal, and a STIC analysis unit 50.

The obtained data are sent from a rearranging unit, which takes the images from the storage unit 41 and performs the rearrangement of the scan planes of the volumetric image on the basis of the time interval defined by the ECG signal detecting unit 51, by the breathing signal detecting unit 52, and by the STIC analysis unit 50.

The contributions of said methods in rearranging the scan planes of the volumetric image are regulated by a combination unit 8 that carries out a weighed sum of the outputs deriving from the rearranging unit 7.

Preferably the combination unit 8 uses image fusion systems of the feature oriented type, which is built for maximizing the displaying of specific effects or characteristics.

Therefore, a three-dimensional model is generated starting from the volumetric image by a reconstructing unit 90, a segmentation unit 91 for recognizing the detected anatomical parts, particularly the walls of the blood vessel, and a 3D model generating unit 92.

Therefore, the three-dimensional model is displayed on a display 14 by rendering by means of an endo-navigation system 6, in particular, a view of the three-dimensional model from a specific point of view is displayed.

The endo-navigation system 6 comprises a system for defining one or more paths of the point of view 60, along which the point of view is movable automatically or under the control of the user.

The path can be established by the user before the real virtual endo-navigation, by a system tracing the path in the three-dimensional model.

Moreover, there is provided a unit calculating the lumen of the blood vessel 63, which therefore measure the width of the vessel for all its extension comprised in the volumetric image.

It is possible to provide an automatic system signaling anomalies, which signals anomalous constrictions beyond threshold values settable by the user or obtainable from databases of reference values or known clinical cases.

A lumen calculation unit 63 further defines the center of the lumen of the blood vessel for each scan plane.

The set of such points for all the scan planes can be used by the a system determining the path of the point of view 60 for defining the automatic movement that has to be followed by the point of view.

There is further provided a unit generating section images 62 from the three-dimensional model.

Such section images are generated along one or more predetermined section planes, preferably two planes, on which the longitudinal axis of the blood vessel at least partially lies and which are perpendicular to each other, and a third transverse plane perpendicular to the two first planes.

Preferably the section images generated are displayed on the display 14 besides the endo-navigation image, and the plane on which the point of view lies is displayed if it is tangent to the plane of the section image.

As an alternative, it is possible to set one or more section planes in relation to the point of view, preferably again three planes perpendicular to each other, and the section images are generated during the movement of the point of view.

It is further possible to display on the display 14 a graph showing the lumen of the vessel in relation to the axial position in the three-dimensional model.

The device further provides a system generating one or more virtual lighting points 61.

The shadows are generated correspondingly to the positioning of the lighting points, which are movable and settable by the user.

The shadows are generated by known shading and rendering systems.

The lighting point can be set a priori after generating the three-dimensional model but before moving the point of view, or it can be moved by the user during the movement of the point of view.

While the invention has been described in connection with the above described embodiments, it is not intended to limit the scope of the invention to the particular forms set forth, but on the contrary, it is intended to cover such alternatives, modifications, and equivalents as may be included within the scope of the invention. Further, the scope of the present invention fully encompasses other embodiments that may become apparent to those skilled in the art and the scope of the present invention is limited only by the appended claims.

The invention claimed is:

1. An imaging method for the cardiovascular system comprising the following steps:
acquiring a volumetric image comprising scan planes adjacent to each other of a periodically moving object;
identifying at least one time interval of at least one periodic movement of said periodically moving object;
rearranging said scan planes of said volumetric image based on said time interval by combining with each other those of the scan planes that are in a same time position with respect to said time interval, wherein said periodically moving object is composed of at least one blood vessel with periodic movements, said periodic movements comprising at least constriction and dilation, and
generating and displaying a virtual endo-navigation three-dimensional model by segmenting and reconstructing said volumetric image,
wherein a point of view is movable along at least one path, for one or more of determining plaques, stenoses or strictures or of analyzing moving structures inside said blood vessel.

2. The imaging according to claim 1, wherein the step of idnetifying at least one time interval of at least one periodic movement occurs by one or more of comparing said scan planes one with the other, contemporaneously acquiring an electrocardiogram signal, or contemporaneously acquiring a breathing signal.

3. The imaging according to claim 2, wherein contributions of comparing said scan planes one with the other, acquiring an electrocardiogram signal, and acquiring a breathing signal in rearranging the scan planes are regulated by a weighed sum.

4. The imaging according to claim 1, wherein said virtual endo-navigation three-dimensional model provides on a reconstructed image one or more virtual lighting points, shadows being generated corresponding to a positioning of said one or more lighting points, said one or more lighting points being movable and settable by a user.

5. The imaging according to claim 1, wherein the step of acquiring is performed in one or more of B-mode, Color Doppler, or other flow displaying mode.

6. The imaging according to claim 1, wherein the point of view of said virtual endo-navigation is automatically movable, said path being predetermined by a user.

7. The imaging according to claim 1, wherein from said three-dimensional model one or more section images along one or more predetermined section planes are generated and displayed, and a lumen of the blood vessel is calculated all along an extension of the blood vessel comprised in the volumetric image.

8. The imaging according to claim 7, wherein the point of view of said virtual endo-navigation is automatically movable, said path being defined by the center of the lumen of the blood vessel all along the extension of the blood vessel comprised in the volumetric image.

9. The imaging according to claim 1, wherein the acquisition of said volumetric image is made by a parallel scanning ultrasound probe.

10. The imaging according to claim 1, further comprising the step of providing an automatic system for signaling anomalies, said automatic system signaling anomalous strictures beyond threshold values settable by a user or obtainable from databases of reference values or of known clinical cases.

11. An imaging device for the cardiovascular system comprising:
a first unit configured to acquire a volumetric image having scan planes adjacent to each other of a periodically moving object;
a second unit identifying at least one time interval of at least one periodic movement of said periodically moving object; and
a system rearranging said scan planes of said volumetric image based on said time interval, said system combining with each other those of the scan planes that are in the same time position with respect to said time interval, wherein said periodically moving object comprises at least one blood vessel having periodic movements, said periodic movements comprising at least constriction and dilation; and
a system generating and displaying a virtual endo-navigation three-dimensional model by segmenting and reconstructing said volumetric image, wherein a point of view is movable along at least one path, thereby determining plaques, stenoses, strictures, or analyzing moving structures inside said blood vessel.

12. The imaging device according to claim 11, wherein said imaging device is configured to operate according to an imaging method comprising:
acquiring said volumetric image comprising said scan planes adjacent to each other of said periodically moving object;
identifying said at least one time interval of at least one periodic movement of said object;
rearranging said scan planes of said volumetric image based on said time interval by combining with each other said scan planes that are in a same time position with respect to said time interval,
wherein said periodically moving object is composed of at least one blood vessel with said periodic movements, said periodic movements comprising at least constriction and dilation, and
generating and displaying said virtual endo-navigation three-dimensional model by segmenting and reconstructing said volumetric image,
wherein said point of view is movable along said at least one path, for determining said plaques, said stenoses, or said strictures or analyzing said moving structures inside said blood vessel.
